(19) United States
(12) Patent Application Publication
(10) Pub. No.: US 2022/0370097 A1
(43) Pub. Date: Nov. 24, 2022

(54) METHODS AND DEVICES FOR DELIVERING IMPLANTABLE PROSTHESES

(71) Applicant: Elixir Medical Corporation, Milpitas, CA (US)

(72) Inventors: MOTASIM SIRHAN, Los Altos, CA (US); JOHN YAN, Los Gatos, CA (US); VINAYAK BHAT, Cupertino, CA (US); JOSEPH PARASCHAC, Campbell, CA (US); BENJAMYN SERNA, Gilroy, CA (US)

(73) Assignee: Elixir Medical Corporation, Milpitas, CA (US)

(21) Appl. No.: 17/568,475

(22) Filed: Jan. 4, 2022

Related U.S. Application Data
(63) Continuation of application No. PCT/US20/40990, filed on Jul. 7, 2020.
(60) Provisional application No. 62/871,916, filed on Jul. 9, 2019.

(51) Int. Cl. A61B 17/34; A61B 17/00

(52) U.S. Cl. CPC A61B 17/3468; A61B 17/00234; A61B 2017/3488; A61B 2017/00243; A61B 2017/00292

(57) ABSTRACT

A system for reshaping a valve annulus includes an elongate template having a length along a longitudinal axis and at least one concavity in a generally lateral direction along said length. The pre-shaped template is positioned against at least a region of an inner peripheral wall of the valve annulus, and at least one anchor on the template is advanced into a lateral wall of the valve annulus to reposition at least one segment of the region of the inner peripheral wall of the valve annulus into said concavity. In this way, a peripheral length of the valve annulus can be foreshortened and/or reshaped to improve coaptation of the valve leaflets and/or to eliminate or decrease regurgitation of a valve.

### CROSS-REFERENCE TO RELATED APPLICATIONS

This application is a continuation of PCT Application No. PCT/US20/40990 (Attorney Docket No. 32016-718.601), filed Jul. 7, 2020, which claims the benefit of U.S. Provisional No. 62/871,916 (Attorney Docket No. 32016-718.101), filed Jul. 9, 2019, the entire contents of which are incorporated herein by reference.

### BACKGROUND OF THE INVENTION

### Field of the Invention

The present invention generally relates generally to medical devices and methods, particularly those in the field of cardiology. More particularly, the present invention relates systems and methods for access heart valves for treatment, repair, or replacement.

Heart valves have important biological function, with a wide range of anatomical configuration including shapes, designs, and dimensions, and are subject to an array of different conditions such as disease conditions that can cause impairment or malfunction. The mitral valve, for example, consists of an annulus containing anterior and posterior leaflets located at the junction between the left atrium and the left ventricle. The valve leaflets are attached to the left ventricle heart papillary muscles via chordae tendineae. Valvular impairment or dysfunction can be caused or exacerbated by changes to the valve configuration including shape, size, and dimension of the valve (or annulus), the length or functionality of the chordae, the leaflets function, causing impairment or dysfunction of the valve.

A variety of cardiac surgical procedures are routinely performed, including for example, surgical annuloplasty, implantation of artificial chordae or repair of chordae, and resection leaflet surgical valve repair. These procedures are performed typically via open heart typically using bypass surgery, including opening the patient's chest and heart, a risky and invasive procedure with long recovery times and associated complications.

As an alternative to such open-heart procedures, less-invasive surgical and percutaneous devices and procedures are being developed to replace or repair the mitral valve. Less invasive surgical and percutaneous options for valve repair typically attempt to replicate more invasive surgical techniques. Many such devices, however, have one or more disadvantages, such as a large size, complex use, limited efficacy, and limited applicability to different anatomical valve configuration.

For these reasons, the results of many percutaneous and less-invasive cardiac procedures, particularly those performed on the mitral valve, have proven to be inferior to open surgical valve repair procedures. Such inferior results often result from limited visualization of the heart valve anatomy during percutaneous and less-invasive cardiac procedures. No single imaging modality provides all anatomical information necessary. Ultrasonic imaging methods do a good job of showing tissue sections, but a poor job of showing the position of the interventional tools in relation to imaged tissue. In contrast, fluoroscopic imaging reveals the tool positions well, but images tissue poorly.

What is needed therefore are devices, tools, systems, and methods for use in or with less-invasive surgical and percutaneous techniques, particularly those performed on a beating heart, and more particularly those performed for mitral valve repair and replacement. Such devices, tools, systems, and methods should preferably address valve regurgitation, minimize or eliminate device migration, be applicable to broader patient population having various valve configurations, while overcoming the limits of current imaging technology. The inventions herein meet at least some of these needs.

### 2. Listing of the Background Art

Commonly owned PCT/US2019/032976 describes systems and methods for reshaping a valve annulus using an elongate template that is attached to the annulus. The full disclosure of this commonly owned application is incorporated herein by reference.

### SUMMARY OF THE INVENTION

The present invention comprises devices and methods for less invasive surgical and/or percutaneous treatment or repair of a body organ, lumen, cavity, or annulus. In a preferred example, the present invention comprises devices and methods for open surgical, less invasive surgical, and percutaneous treatment or repair of heart valves comprising valve annulus and valve leaflets. An example of heart valves comprises aortic, mitral, pulmonary, and tricuspid valves. Although certain examples show a specific valve, the inventions described and claimed herein are applicable to all valves in the body and additionally other body annulus, lumen, cavity, and organs.

In one example, an elongate device has one or more probe elements, such as whiskers, flaps, feelers, wires, sensors, or the like, extending from an engagement end or region near a portion of the device. The probe elements typically extend outwardly and distally relative to a central axis of a shaft or other body of the elongate device. Probe elements may be formed from any materials capable of engaging and being deflected by tissue during a surgical procedure. Suitable polymers include pebax, nylon, abs, ePTFE, or the like, hydrogels, metals or composite materials. They may be constructed with radiopaque additives including barium sulfate, bismuth subcarbonate, bismuth oxychloride, tungsten, or the like. They may have radiopaque markers disposed along their length, including platinum bands, radiopaque inks, or sections of polymers with radiopaque additives. They may be constructed with echogenic features including hollow glass beads, air pockets, or two or more materials of different stiffness or density. They may be constructed with echogenic surface finishes which may include bead blasting, surface texturing, or retro-reflective textures (including hemispheres or corner cube shapes). Dimensions of the probe elements will be dependent on the exact application but will generally have a thickness between 0.1 mm and 1 mm, a width between 0.5 mm and 2 mm, and a length between 1 mm and 10 mm.

In further examples, the probe elements may contain radiopaque material(s), for example in the form or strips, layers, features, patterns, and the like, to enhance their fluoroscopic visibility. In still further examples, the probe elements may contain echogenic features to improve their visibility to ultrasonic imaging techniques. For example, the echogenic features may include one or more of the following: retroreflective surface textures, air bubbles, hollow glass beads, closed cell foam structures, or a mix of materials with significantly different stiffnesses. In preferred examples, the probe elements will incorporate both radiopaque materials and echogenic features.

In some examples, the probe elements are attached to a sheath. In other examples, the probe elements are attached to a therapeutic, diagnostic, locating/positioning or marking device that passes through a sheath. In other examples, the probe elements are attached to an implantable device. In further examples, the implantable device is a helical anchor that couples with a target tissue. In still further examples, the probe elements are configured and arranged to hold or stabilize the implantable device in apposition to the tissue while the tissue heals after implantation. In still further examples, the probe elements are configured and arranged to be held in apposition to the tissue by the implantable device while the tissue heals after implantation.

In some examples, the target tissue comprises a mitral valve annulus. In other examples, the target tissue comprises an aortic valve annulus. In still other examples, the target tissue comprises a tricuspid valve annulus. In yet other examples, the target tissue comprises a pulmonary valve annulus, and in further examples, the target tissue comprises one or more venous valves.

The probe elements may interact with the target tissue in a variety of ways, for example by deflecting in response to engaging the target tissue. In other examples, the probe elements interaction may comprise electrically contacting, coupling, or sensing with the target tissue. In still other examples, the probe elements may be configured to vibrate, oscillate, or otherwise move when out of tissue contact, e.g. in response to blood or other fluid flow, an applied current, or other stimulation. In such cases, tissue contact can be detected when the probe elements stop moving when in response to tissue contact.

In additional examples, the probe elements are attached to the elongate device and configured to interact with the target tissue in a manner which indicate distances between the target tissue and a location on the elongate device. In a further example, the probe elements interact with the target tissue differently as the distance between the elongate device and the probe elements is increased or decreased. In other examples, different individual probe elements or sets of probe elements interact differently with the target tissue depending on the distance between the target tissue and the elongate device, e.g. longer probe elements may deflect in response to engaging tissue sooner than shorter probe elements; probe elements oriented at particular angles relative to the elongated device may deflect in response to engaging tissue sooner than other probe elements; probe elements having different shapes (linear, non-linear, sinusoidal, bifurcated, trifurcated, etc.) may deflect in response to engaging tissue at times different than shorter probe elements.

In some examples, the elongate device with probe elements may traverse the venous system. In other examples, the elongate device with probe elements may traverse the inferior vena cava. In a further example, the elongate device with probe elements crosses the septum between the right atrium and the left atrium. In still further examples, the elongate device with probe elements crosses the septum between the right atrium and the left atrium in the region of the fossa ovalis.

In some examples, the elongate device with probe elements traverses the arterial system. In further examples, the elongate device with probe elements traverses the aorta. In yet further examples, the elongate device with probe elements enters the left ventricle. In still further examples, the elongate device with probe elements cross from the left ventricle to the left atrium. In other examples, the elongate device with probe elements crosses from the left ventricle to the left atrium between the leaflets of the mitral valve.

In a first aspect, the present invention provides apparatus in the form of a surgical locating tool. The surgical locating tool may be used in a variety of surgical procedures, particularly less-invasive and percutaneous surgical procedures where visual access is limited, typically relying on fluoroscopy, ultrasound, optical coherence tomography (OCT), optical cameras, and the like. The surgical locating tools of the present invention can provide positional feedback as the locating tool approaches and engages a target location on a patient tissue site, often where the target location cannot be adequately visualized using external visioning capabilities. In particular, the positional feedback may be provided by one or more probe elements on the surgical locating tool, as will be described in more detail below.

An exemplary surgical locating tool constructed in accordance with the principles of the present invention comprises a shaft having one or more probe elements coupled thereto. The shaft typically has an engagement end, and the shaft will usually be configured to deliver and implant or to engage in interventional tool against an internal tissue surface. The one or more probe elements may be coupled or otherwise configured to extend outwardly from the engagement end of the shaft, and the probe elements are typically configured to detectably deflect when engaged against or in proximity with the internal tissue surface.

In some instances, the probe elements may be configured to be imaged by medical imaging devices, including any of fluoroscopic, ultrasonic, OCT, or other optical imaging systems of type commonly employed in performing such less-invasive or percutaneous surgical procedures. More specifically, the probe elements may be radiopaque, typically incorporating or attached to radiopaque markers, so that they are imageable under fluoroscopy. Alternatively, the probe elements may be acoustically opaque to enhance imaging under ultrasound observation. In still other instances, the probe elements may be optically visible using optical imaging sensors, such as viewing by cameras, CCD's, and the like, placed on other devices proximate the issue target sites. In still further instances, deflection may be detected by sensors attached to the probe elements, such as stress sensors, strain sensors, position encoders, and the like.

In still other instances, the shaft of the surgical locating tool of the present invention will be configured to deliver an implant to the target tissue site. For example, the shaft may have a channel which extends or opens to the engagement end of the shaft. The channel may be a receptacle or other cavity which extends only part way through or into the shaft. In most instances, however, the channel will extend an entire length of the shaft so that an implant may be delivered through the shaft after the engagement end has been located adjacent the target surgical site.

In yet other instances, the channels or other features of the shaft may be configured to position an interventional tool, such as an electrosurgical device, a tissue ablation device, a tissue resection device, or the like. In such instances, the shaft may be configured to position a separate interventional tool or, alternatively, the shaft may itself incorporate the interventional tool, i.e., the interventional tool or device may be integrated with locating tool to incorporate an interventional capability.

In certain instances, the locating tools of the present invention will have a plurality of probe elements at the engagement end of the shaft, typically from 2 to 24 probe elements. The probe elements may be arranged symmetrically or asymmetrically about an axial center line of the shaft. The probe elements may have the same or different lengths. The probe elements may have the same or different shapes. The probe elements may be arranged collectively to taper radially outwardly in a direction away from the engagement end of the shaft, e.g., may be arranged in a generally conical configuration with a large end of the cone spaced away from the engagement end of the shaft. In still other instances, the probe element(s) may be oriented at the same or different angles relative to the axial center line of the shaft, where the angles may vary from proximal end or section of the probe element in a direction toward a distal end or section of the probe element. The probe elements may have a constant cross-sectional area or shape or may have cross-sectional areas or shapes which vary along their lengths. Another instances, the probe elements may be configured to deflect primarily at a base end where they are attached to the shaft or may be configured to have a distributed deflection along their lengths.

In a second aspect, the present invention provides methods for locating and modifying an internal tissue surface of a patient. The methods comprise engaging one or more probe elements on or near an engagement end of a shaft against a target location on the internal tissue surface. Deflection of one or more of the probe elements is then observed to determine a position of the engagement end of the shaft relative to the target location. A tissue-modifying event may then be initiated when the engagement end of the shaft is at a desired position relative to the target location on the tissue.

In specific instances, observing the probe elements may comprise at least one of fluoroscopic imaging, ultrasonic imaging, and optical imaging. In the case of fluoroscopic imaging, the one or more probe elements may be radiopaque, partially radiopaque, or include radiopaque elements or markers disposed along the length of the probe element. In the case of ultrasonic imaging, the one or more probe elements may be acoustically opaque, reflective, or echogenic. In the case of optical imaging, the probe elements may be imaged by a camera on a tool near the location of the target tissue. In other instances, the probe elements may be imaged by OCT, or other surgical imaging methods. As an alternative to imaging, deflection of the probe elements may be detected using motion sensors attached or coupled to the probe elements, such as stress transducers, strain transducers, position encoders, and the like.

Initiating a tissue-modifying event may comprise delivering an implant from the shaft to , tissue at or near the target location. For example, the implant may comprise a plication tip or other element intended to be implanted on a heart valve annulus, such as a mitral valve annulus.

In other instances, initiating the tissue-modifying event may comprise positioning the shaft to engage an interventional tool against the target tissue. For example, the interventional tool may be advanced through the shaft to a position near the engagement end of the tool which is being held proximate the target tissue location. In other instances, the interventional tool may be integrated with the shaft of the locating tool.

In further instances of the methods of the present invention, a plurality of probe elements will be engaged against the target location on the internal tool surface. The plurality may comprise two or more probe elements, typically being in a range from 2 to 24 probe elements. The probe elements may be arranged symmetrically or asymmetrically about a center line of the shaft. The probe elements may all have the same length or may have different lengths. The probe elements may comprise longer probe elements and shorter probe elements, typically being interdigitated or otherwise interspersed with each other to engage tissue at different times or at different positional locations of the shaft. The probe elements may taper radially outwardly in a distal direction away from the engagement end of the shaft, for example in a radially outward conical pattern. The shapes of the probe elements may vary, including linear, nonlinear, serpentine, and the like. The probe elements may deflect radially outwardly from a center line of the probe shaft at similar angles or different angles. The probe elements may have a constant cross-sectional shape or area, or the cross-shape or area may vary among different individual or groups of probe elements. The probe elements may be configured to deflect primarily at a base end where they are attached to the shaft, e.g., the base end may act as a pivot or fulcrum for deflection of the probe element. In other instances, the probe elements may be flexible along their lengths and configured to deflect in a distributed manner between the base and detached to the shaft and a distal end in free space.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an elongate device with probing elements extending distally and outward. Depending on the width of the probe elements, they can be considered flaps that have greater stiffness and will be considered as probe elements hereafter.

FIG. 2 shows an end view of the elongate device with probing elements and an integral central channel

FIG. 3 shows an elongate device with probing elements extending distally in line with the device.

FIG. 4 shows a side view of the elongate device in FIG. 1.

FIG. 5 shows the elongate device of FIG. 1 approaching a tissue wall at an angle, the tissue wall having a movable tissue segment, for example, a valve leaflet.

FIG. 6 shows the elongate device of FIG. 5 in contact with the wall, with the probing elements deflected and in contact with the target tissues.

FIG. 7 shows the elongate device of FIG. 6, with the probing elements remaining in contact with the movable tissue segment as it moves

FIG. 8 shows an end view of an elongate device having probe elements of varying lengths.

FIG. 9 shows a side view of an elongate device having probe elements extending primarily outward from the elongate device.

FIG. 10 shows an end view of an elongate device having probe elements with variable cross section. As shown, the section is thinner and therefore more flexible near the elongate device, creating a hinge effect.

FIG. 11 shows an end view of an elongate device having probe elements with variable cross section. As shown, the section reduces in width or thickness toward the tips of the probe elements, creating a less traumatic, more flexible tip on one or more probe elements.

FIG. 12 shows an end view of an elongate device having probe elements connected at the ends by bridging segments.

FIG. 13 shows an end view of an elongate device having probe elements with a branching structure on at least some of the probe elements.

FIG. 14 shows a side view of an elongate device having probe elements with an angle relative to the elongate device that changes along the length of the probe element.

FIG. 15 shows a side view of an elongate device having probe elements which branch to create a probe segment directed inward and distally.

FIG. 16 shows a side view of an elongate device having probe elements which branch to create a probe segment directed inward and proximally.

FIG. 17 shows two adjacent connected probe elements, the connection having a shape that allows the connection to partially fold so that the connection and the probe elements can move inwardly to a smaller diameter.

FIG. 18 shows an elongate device with probe elements in section view, with an anchoring device through the center channel. The anchoring device is coupled to the target tissue.

FIG. 19 shows an elongate device with probe elements, and anchoring device coupled to the tissue, and a tissue shaping template coupled to the anchoring device.

FIG. 20 shows an elongate device with an array of probe elements arranged along its length.

FIG. 21 shows an elongate device with probe elements having a solid center support.

FIG. 22 has six panels showing alternative cross-section sectional geometries for the elongate device.

FIGS. 23A and 23B show an elongate device with probe elements, the elongate device having an internal structure that allows the height or diameter of the device to change by moving one member proximally or distally relative to the other member.

FIGS. 24A and 24B show an elongate device having probe elements that connect at the distal end to form a basket. Moving one end of the basket proximally or distally relative to the other end adjusts the diameter of the basket.

FIG. 25 shows a simplified elongate device having two probe elements, the elongate device configured to rotate about an axis to change the orientation of the two probe elements relative to the target tissue.

FIG. 26 shows an elongate device having two probe elements composed of at least two distinct materials.

FIGS. 27A and 27B show an elongate device having probe elements and an outer sheath. Moving the probe elements proximally or distally relative to the outer sheath adjusts the effective length of the probe elements.

FIGS. 28A-28C show a system of nested elongate devices with probe elements of different lengths, and a tissue coupling anchor to be delivered to the target tissue.

FIG. 29 shows an elongate device with multiple independent probe elements, one or more of which can be moved proximally and distally relative to one or more of the others.

### DETAILED DESCRIPTION OF THE INVENTION

The phrase “valve annulus” as used herein and in the claims means a ring-like tissue structure surrounding the opening at base of a heart valve that supports the valve's leaflets. For example, the annulus of the mitral valve, the tricuspid valve, the aortic valve, the pulmonary valve, venous valves and other annuluses of valves in the body. In the mitral valve, the annulus typically is a saddle-shaped structure that supports the leaflets of the mitral valve.

The phrase “peripheral wall” as used herein and in the claims as applied to a valve annulus means a surface or portion of the tissue of the valve annulus, and/or a portion of the tissue adjacent to the valve annulus.

“Concavity” as used herein and in the claims means a depression or well formed in a surface of the template. The concavity may comprise flat regions joined at angles, e.g. being rectilinear, but will more typically have a curved bottom portion joining a pair of generally straight and/or curved walls or legs. The curved bottom portion will typically span an arc of at least 45°, often at least 60°, usually at least 90°, typically at least 135°, and sometimes spanning a full 180°, with exemplary ranges from 45° to 180°, from 60° to 180°, from 60° to 135°, and from 90° to 135°. The concavities of the present invention will typically be symmetric having opposed walls or legs on each side of a central axis. In other cases, however, a concavity may be asymmetric with walls or legs on each side having unequal lengths and in sometimes having only a single wall. Examples of concavities include the inner surface of a circle or sphere or other.

“Convexity” as used herein and in the claims means a curved surface on the template like an exterior of a circle, parabola, ellipse, or the like. A convexity will typically be formed on a surface of the template on the side opposite to that of a concavity, and vice versa. Examples of convexities include the outer surface of a circle or sphere or other.

As used herein and in the claims, an “implant” means an article or device that is introduced into and left in place in a patient's body by surgical methods, including open surgery, intravascular surgical methods, percutaneous surgical methods, and least invasive or other methods. For example, aortic valve replacement implant, coronary stent implant, or other types of implants.

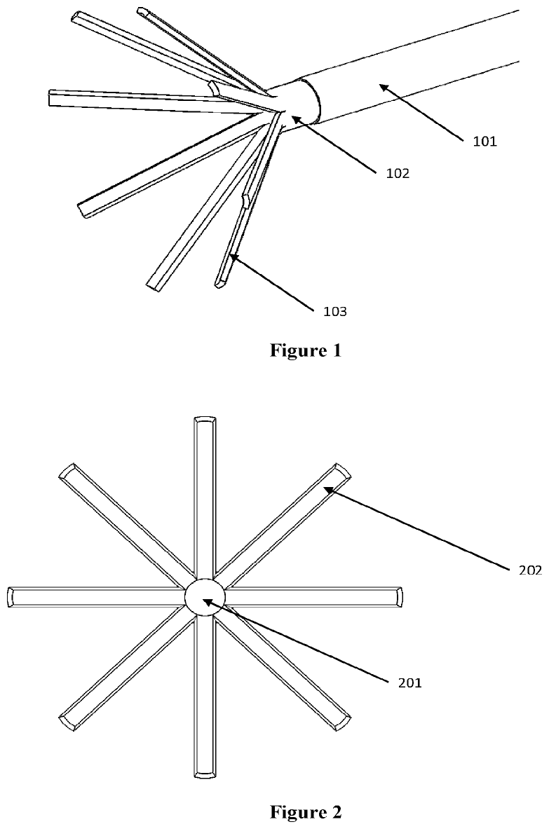

As shown in FIG. 1, an elongate device 101 has a probe element junction 102 extending into one or more probe elements 103. In FIG. 1, eight probe elements 103 extend distally and outward from the elongate device 101, however other shapes and numbers of probe elements may be advantageous.

Probe elements can be formed by, for example, by insert molding one or more probe elements together and attaching to the elongate device, by laser cutting a tube formed of probe element material, or by cutting the desired probe element pattern in a flat and shaping it (if needed) to fit the elongate device, by photochemical etching, or a combination of these processes. The probe elements can be shaped during processing (particularly in the case of the injection molded variants), bent to shape after cutting, or heat set to final shape in post processing. Additional features, such as hinge points, sensors, conductive pads, or wires, can be attached by processes including bonding, welding, crimping, and the like.

FIG. 2 shows an end view of the elongate device 101 from FIG. 1, showing the inner surface 202 of the probe elements, and a central channel 201. The central channel 201 may be used for delivery of a therapeutic or diagnostic device or material to a target tissue site. An implant may be placed through the central channel 201. The central channel 201 may also be used to place a marker into the tissue, or inject a contrast solution for imaging tissue, lumens, or body cavities adjacent to the probe elements. The central channel 201 may also be used to biopsy or remove target tissue.

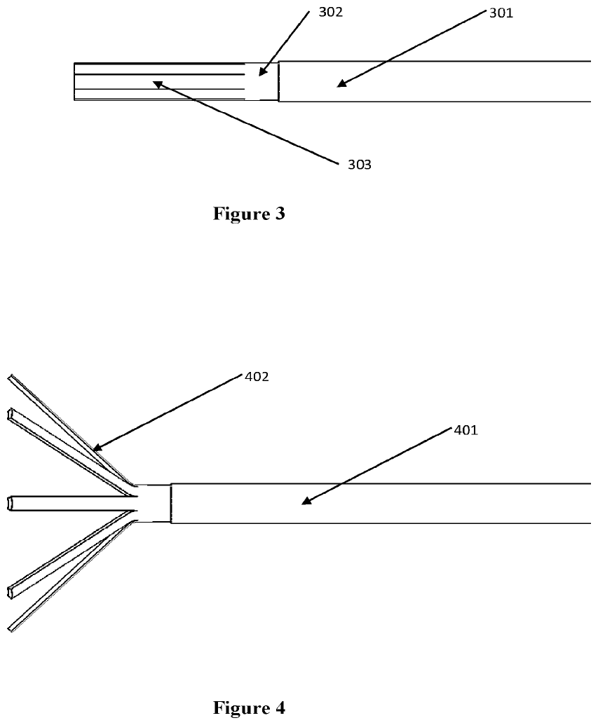

FIG. 3 shows an elongate device 301 having a probe element junction 302 and one or more probe elements 303 extending distally in the direction of the elongate device 301. Probe elements 303 in this configuration are deliverable through a channel the same size as the elongate device 301. It may be advantageous to form probe elements 303 in this configuration, or to temporarily constrain outwardly directed probe elements (for example, probe elements 202 shown in FIG. 2) in this configuration for delivery to the target tissue site.

FIG. 4 shows a side view of an elongate device 401 having one or more probe elements 402 which extend outward and distally from the elongate device at an angle of approximately 45 degrees.

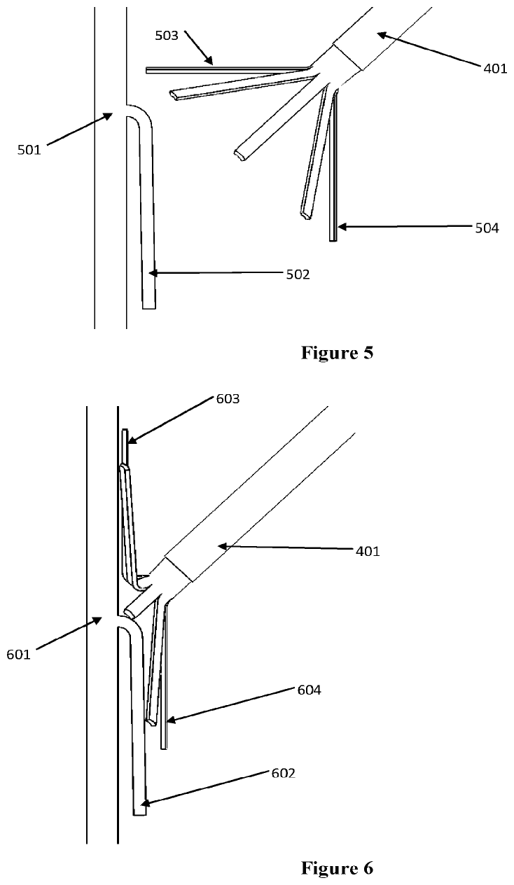

FIG. 5 shows the elongate device 401 of FIG. 4 approaching target tissue 501 at approximately a 45 degree angle. At this angle, the top probe element 503 approaches target tissue at approximately a right angle, and the bottom probe element 504 is approximately parallel to the target tissue. A mobile segment 502 of the target tissue is approximately parallel to the target tissue.

FIG. 6 shows the elongate device 401 of FIG. 4 approximating target tissue 601 at approximately a 45 degree angle. When elongate device 401 and target tissue 601 are held in approximation, the top probe element 603 deflects to extend upward approximately parallel to the target tissue, and the bottom probe element 604 remains directed downward approximately parallel to the target tissue. A mobile segment 602 of the target tissue is approximately parallel to the target tissue.

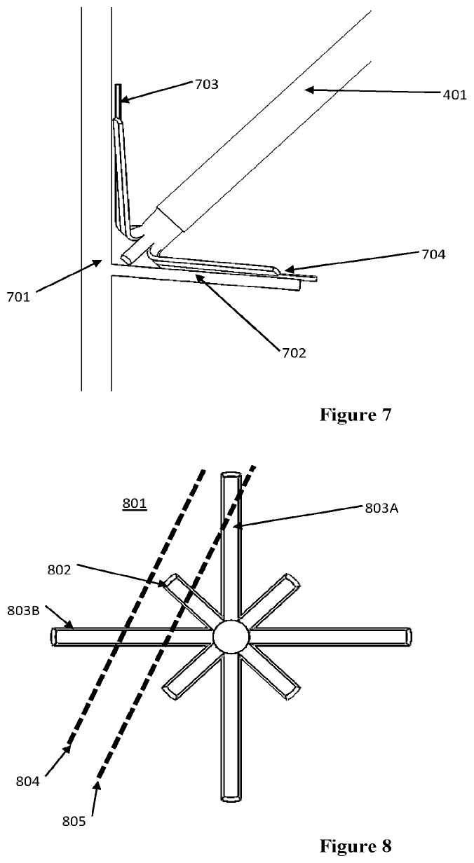

FIG. 7 shows the elongate device 401 of FIG. 4 approximating target tissue 701 at approximately a 45 degree angle. In this figure, the mobile segment 702 of the target tissue has moved to a position approximately perpendicular to the target tissue 701. When elongate device 401 and target tissue 701 are held in approximation, the top probe element 703 remains deflected upward approximately parallel to the target tissue, and the bottom probe element 704 deflects along with the mobile tissue segment 702. In this configuration, images showing the motion of the probe element 704 can be used to infer motion of the mobile segment 702 of the target tissue. Images showing the motion of the probe element 704 can also be used to infer the location of the elongate device 401 relative to the mobile segment 702 of the target tissue.

FIG. 8 shows and end view of an elongate device 801 having one or more short probe elements 802 and one or more long probe elements 803A-B. In use, the long probe elements 803A-B will move with moving tissue at a first distance (shown by line 804) from the elongate device 801, while the short probe elements 802 will not be affected by tissue motion at said first distance. When the elongate device 801 is moved closer to the moving tissue, to a second distance (shown by line 805) that is less than said first distance, both the short probe elements 802 and the long probe elements 803A and 803B will be affected by tissue motion. Similarly, probe elements of 3 different lengths, 4 different lengths, or more could be used to indicate position of the elongate device relative to moving tissue. Probe elements can interact with stationary tissue features (for example, a luminal opening in a wall) in a similar fashion, with long probe elements 803 reacting first to the stationary tissue feature, and short probe elements 802 reacting only when the elongate device 801 is moved closer to the stationary tissue feature.

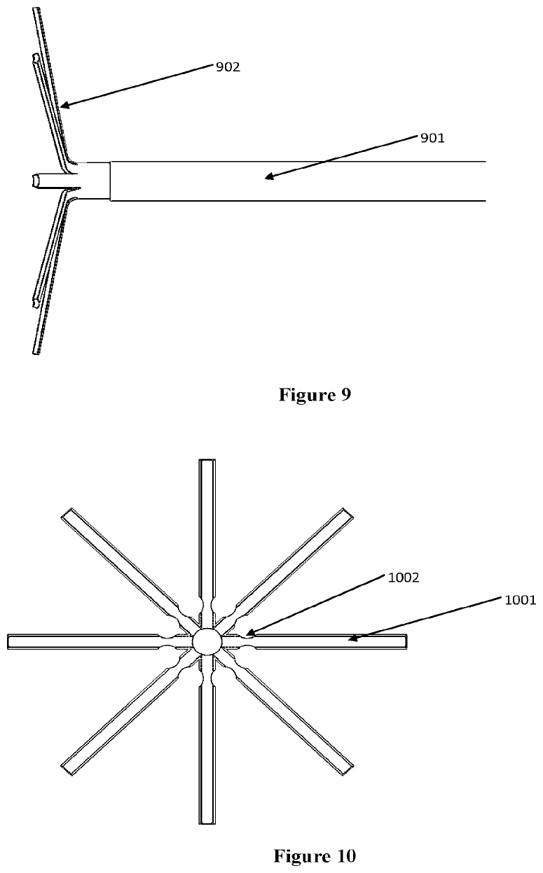

FIG. 9 shows an end view of an elongate device 901 having one or more probe elements 902 extending outwardly from the elongate device 901. The angle between the probe elements 902 and the long axis of the elongate device 901 approaches perpendicular. In this configuration, the probe elements 902 would move in response to bumps or curves in the surface of a target tissue.

FIG. 10 shows an end view of an elongate device having one or more probe elements 1001 with a varying cross section. As shown, there is a reduced cross section 1002 near the junction of the probe elements 1001 and the elongate device. This reduced cross section 1002 creates a more flexible “hinge” region, offering increased control over the shape the probe elements 1001 take when interacting with target tissue.

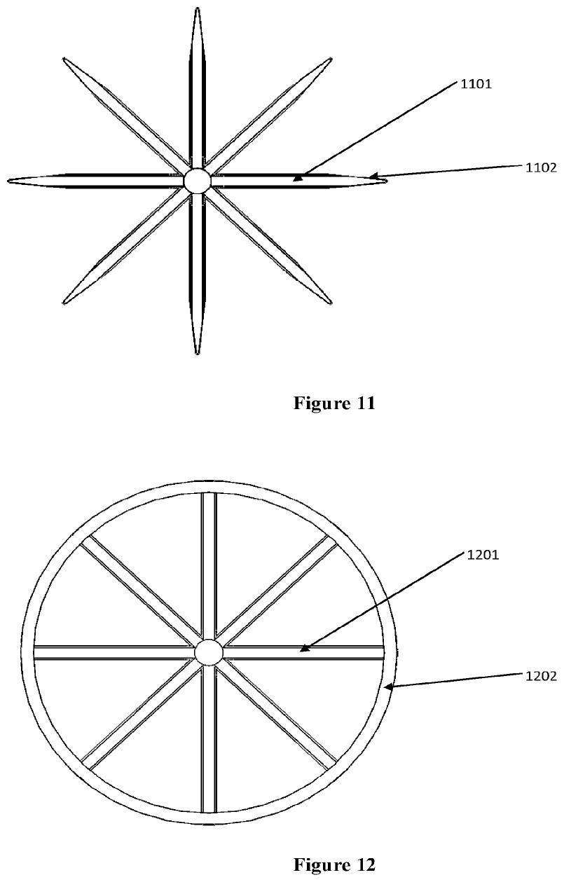

FIG. 11 shows an end view of an elongate device having one or more probe elements 1101 with a varying cross section. As shown, there is a reduced cross section 1102 near the distal end of the probe elements 1101. This reduced cross section 1102 creates a more flexible tip region, offering increased control over the shape the probe elements 1101 take when interacting with target tissue.

FIG. 12 shows an end view of an elongate device having two or more probe elements 1201 with one or more branches 1202 extending from one or more of the probe elements 1201 and connecting to one or more of the adjacent probe elements 1201. As shown, the branches 1202 extend from the ends of each of the eight probe elements 1201 and connect each of the adjacent probe elements 1201 to form a continuous ring. It may be advantageous to have the branches 1202 extend from a location proximal to the tip of the probe elements 1201, or to connect subsets of probe elements 1201, leaving others unconnected.

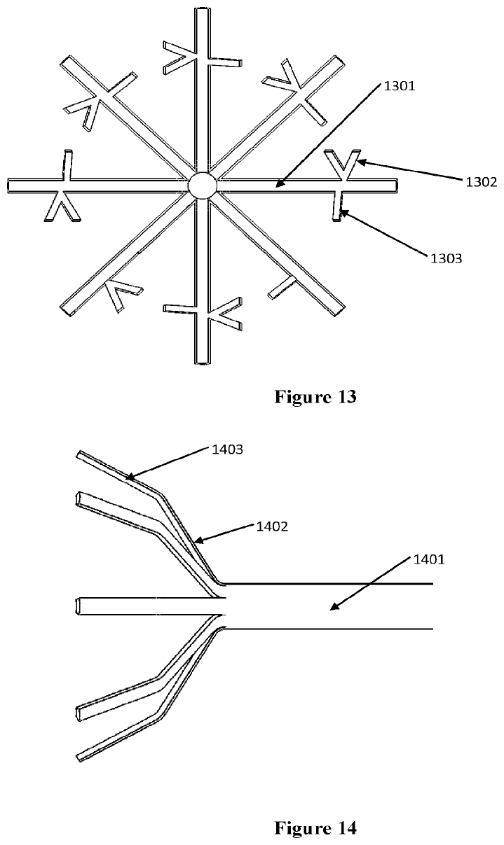

FIG. 13 shows an end view of an elongate device having one or more probe elements 1301 having forked branches 1302 and single branches 1303 extending from one or more of the probe elements. Each probe element 1301 can have no branches, one or more forked branches 1302, one or more single branches 1303, or a combination of forked branches 1302 and single branches 1303. The forked branches 1302 and single branches 1303 can extend substantially planar to the probe elements 1301 or be deflected inward or outward from the probe elements 1301 relative to the elongate device.

FIG. 14 shows an elongate device 1401 having one or more probe elements having a proximal segment 1402 extending from the elongate device 1401 at a first angle, and a distal segment 1403 of the probe elements extending from the proximal segment 1402 at a second angle relative to the long axis of the elongate device 1401. As shown, the second angle is a shallower angle relative to the elongate device 1401 compared to the first angle. Probe elements having proximal segments 1402 and distal segments 1403 at different angles will interact with target tissues in different ways than straight probe elements, offering different information about the location and motion of the target tissue than a straight probe element would. It may be further advantageous to combine both straight probe elements and probe elements having proximal segments 1402 and distal segments 1403 at different angles in the same elongate device.

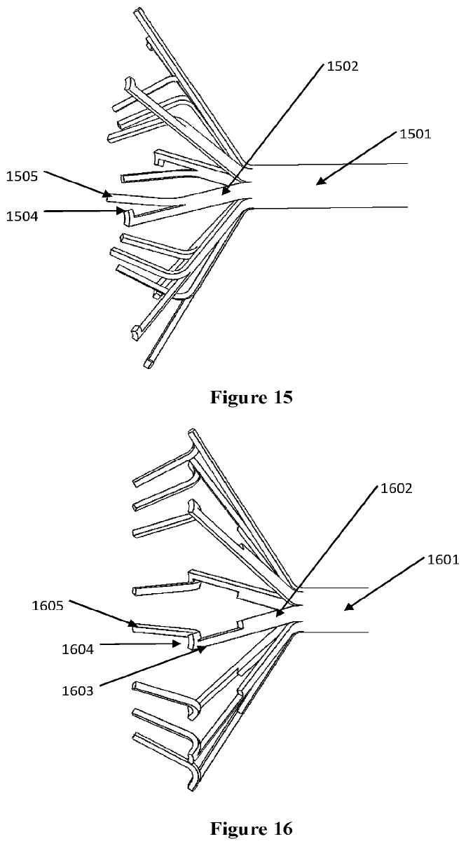

FIG. 15 shows a side view of an elongate device 1501 having one or more probe elements 1502 disposed at a first angle relative to the long axis of the elongate device 1501 with one or more branches 1505 disposed at a second angle relative to the long axis of the elongate device 1501. As shown, the branches 1505 extend distally and inwardly from the branching point. It may be advantageous for the branches 1505 to extend distally and outwardly from the branching point, or remain in substantially the same plane as the probe element 1502.

FIG. 16 shows a side view of an elongate device 1601 having one or more probe elements 1602 disposed at a first angle relative to the long axis of the elongate device 1601 with one or more branches 1605 disposed at a second angle relative to the long axis of the elongate device 1601. As shown, the branches 1605 extend proximally and inwardly from the branching point. It may be advantageous for one or more branches 1605 to extend proximally and outwardly from the branching point, or remain in substantially the same plane as the probe element 1602.

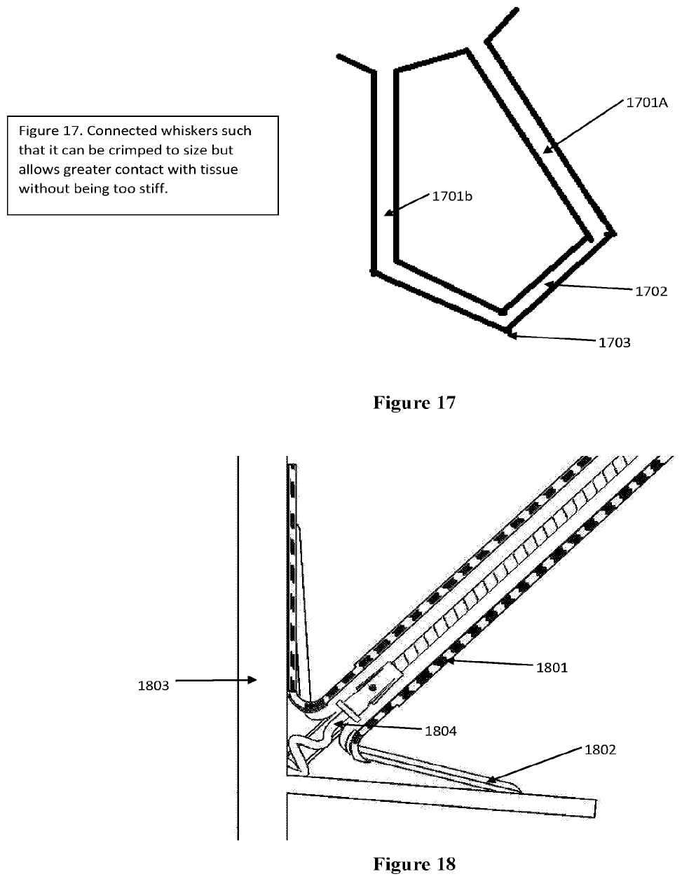

FIG. 17 shows two adjacent probe elements 1701A and 1701B, connected by a connecting branch 1702 having a bend 1703. The bend 1703 can fold as the probe elements 1701A and 1701B move relative to each other. In a small diameter delivery configuration, the connecting branch 1702 will be folded on itself at the bend 1703, and the probe elements 1701A and 1701B will approximate each other, allowing the assembly to pass through a catheter appropriately sized for access to the target body area.

FIG. 18 shows a section view of an elongate device 1801 having one or more probe elements 1802 in contact with target tissue 1803. With the location of the elongate device 1801 relative to the target tissue 1803 verified by imaging methods aided by the one or more probe elements 1802, a tissue coupling anchor 1804 is placed through the center channel of the elongate device 1801 and coupled to the target tissue in the desired location. The probe elements 1802 in combination with various imaging modalities can be used to enhance visibility of the target tissue 1803 and aid in placing the tissue coupling anchor 1804 accurately relative to the target tissue 1803.

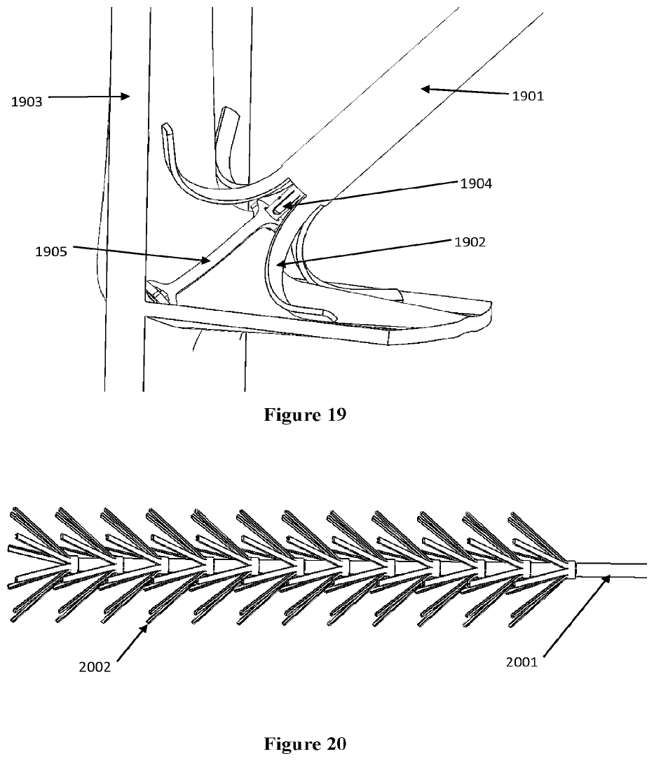

FIG. 19 shows an isometric view of an elongate device 1901 having one or more probe elements 1902 in contact with target tissue 1903. Disposed within the center channel of the elongate device 1901 is a tissue coupling anchor 1904. Coupled to the tissue coupling anchor is a tissue shaping template 1905, which reshapes the target tissue to a desired configuration. The probe elements 1902 in combination with various imaging modalities can be used to enhance visibility of the target tissue 1903 and aid in placing the tissue shaping template 1905 in the correct rotational orientation relative to the target tissue. After placement of the tissue shaping template 1905, the probe elements 1902 can be used to verify the re-shaping and motion of the tissue are within target parameters.

FIG. 20 shows an elongate device 2001 having an array of probe element groups 2002 disposed along its length. The displacement and motion of the individual probe element groups 2002 can be used to locate certain tissue features relative to the long axis of the elongate device 2001, or to locate more than one tissue feature relative to another tissue feature.

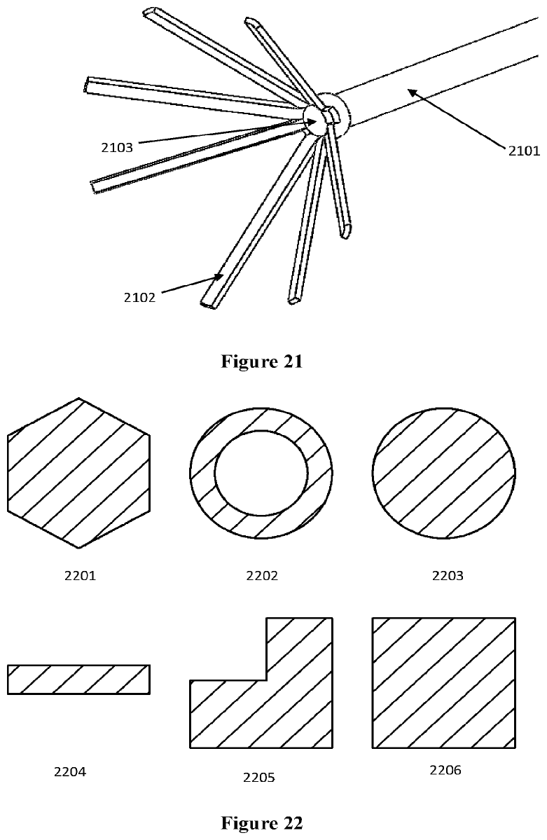

FIG. 21 shows an elongate device 2101 having one or more probe elements 2102 and a solid central cross section 2103. The cross section 2103 can be optimized to make the profile of the elongate device 2101 as small as possible to access small lumens, or to fit alongside other instruments.

FIG. 22 includes six panels showing a series of possible cross-section geometries for an elongate device in accordance with the present invention including polygonal 2201, tubular 2202, circular 2203, flat 2204, cutout 2205, and square 2206. Closely related shapes, for example polygons with different numbers of sides, tubes with multiple lumens, ellipses, arcuate segments, rectangles, etc. may also have advantages as cross sections for elongate devices.

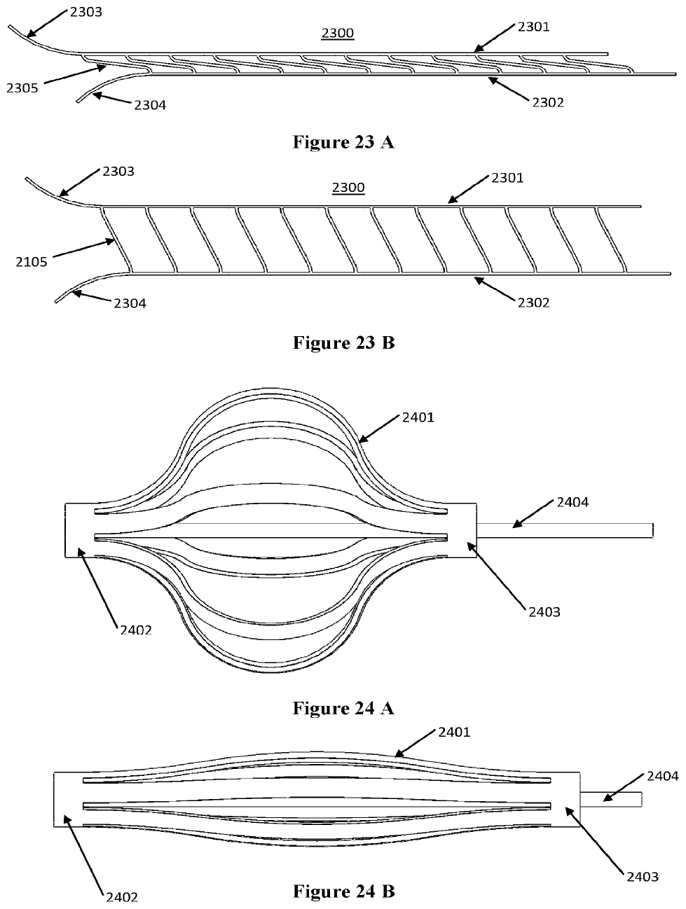

FIGS. 23A and 23B show an elongate device 2300 with a variable height or diameter. The elongate device 2300 comprises a first elongate segment 2301 having one or more probe elements 2303 and a second elongate segment 2302 having one or more probe elements 2304, the two elongate segments 2301 and 2302 being connected by an array of rungs 2305 such that by moving the elongate segments 2301 and 2302 proximally or distally relative to each other, the separation distance between them, and therefore the height or diameter, can be change. FIG. 23A shows this device in the narrow, small diameter, short configuration, and FIG. 23B shows this device in the wide, large diameter, tall configuration.

FIGS. 24A and 24B show an elongate device with one or more probe elements 2401 joined to a distal hub 2402 and a proximal hub 2403, the distal hub being coupled to a shaft 2404, and the proximal hub being slidably engaged to the shaft 2404. When the hubs 2402 and 2403 are brought closer together, the probe elements 2401 bend more and have an increased diameter (as shown in FIG. 24A). When the hubs 2402 and 2403 are moved farther apart, the probe elements 2401 straighten, and have a decreased diameter (as shown in FIG. 24B). This changeable diameter can be used to visualize the shape and size of body lumens, pockets, aneurysms, or other tissue features.

FIG. 25 shows an elongate device 2501 having one or more probe elements 2502 which can be rotated 2503 clockwise or counterclockwise around an axis 2504.

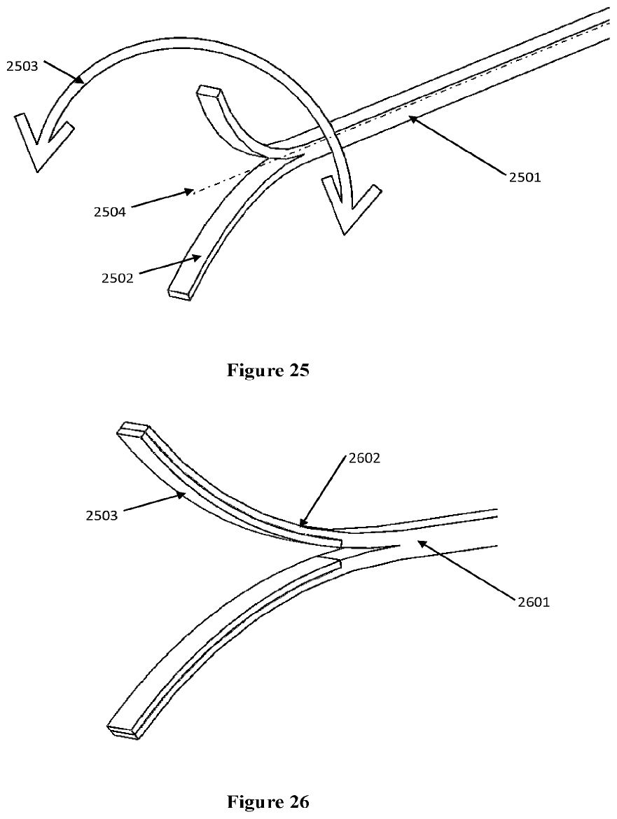

FIG. 26 shows an elongate device 2601 having one or more probe elements 2602 with a secondary feature 2503. The secondary feature can be a second material with enhanced imaging properties (for example, an echogenic layer), or a sensing element with a connection extending back through the elongate device 2601 (for example, an pressure sensor, a strain sensor, a piezoelectric material, microphone, oxygen sensor, electrode, or other similar sensing equipment). Such a sensor may offer additional information to the user, for example the blood oxygenation at the probe element 2602 could indicate if it is in the venous or arterial blood system.

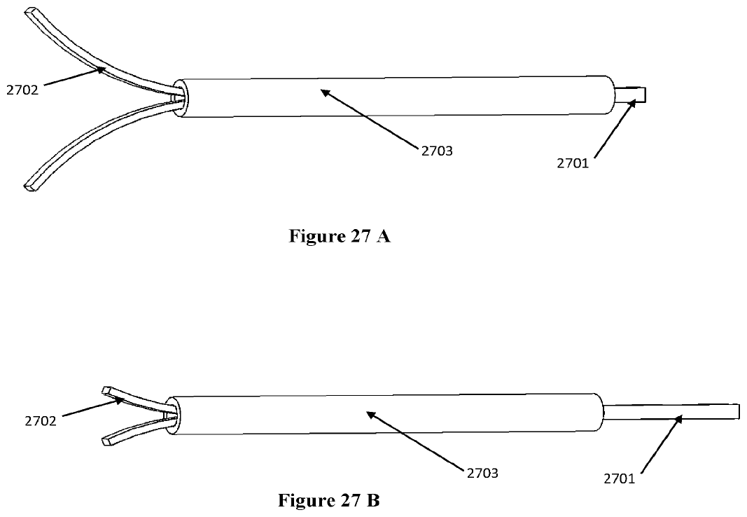

FIGS. 27A and 27B show an elongate device 2701 having one or more probe elements 2702 and a slidable sleeve 2703 disposed around the elongate device 2701. FIG. 27A shows the slidable sleeve 2703 retracted proximally relative to the probe element 2702, with a first length of probe element 2702 extending distally from the slidable sleeve 2703. In this configuration, the probe elements 2702 could be used to guide the elongate device 2701 to the general vicinity of the target tissue.

FIG. 27B shows the slidable sleeve 2703 extended distally relative to the probe element 2702, with a second length of probe element 2702 extending distally from the slidable sleeve 2703. This second length is shorter than the first length illustrated in FIG. 27A. In this configuration, the probe elements 2702 could be used to guide the elongate device 2701 to the target tissue with greater precision than the configuration shown in FIG. 27A.

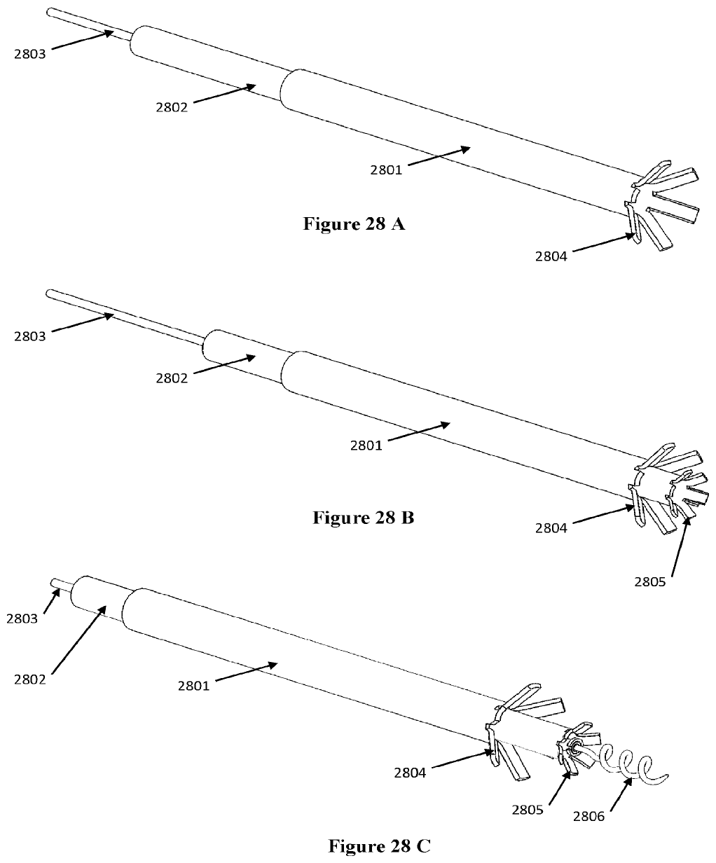

FIGS. 28A-28C show an elongate device consisting of an outer sheath 2801, and inner sheath 2802 and an anchor shaft 2803 coupled to a tissue coupling anchor 2806. FIG. 28A shows the device of FIG. 28 with the outer sheath 2801 covering both the distal end of the inner sheath 2802 and the tissue coupling anchor 2806. The outer sheath 2801 has one or more probe elements 2804 having a first length. FIG. 28B shows the device of FIG. 28 with the inner sheath 2802 moved distally relative to the outer sheath 2801 so that the distal end of the inner sheath 2802 extends distally from the distal end of the outer sheath 2801. The inner sheath 2802 has inner probe elements 2805 having a second length on the distal end of the inner sheath. The second length of the inner probe elements 2805 is different than the probe elements 2804 of the outer sheath 2801. The different length elements can be used to resolve different size features. In addition, the longer probe elements can be used to approach the vicinity of the target tissue, and the shorter probe elements can be used to refine that positioning. FIG. 28C shows the tissue coupling anchor 2806 extending distally from both the inner sheath 2802 and the outer sheath 2803. In this configuration, the tissue coupling anchor can be coupled to the target tissue.

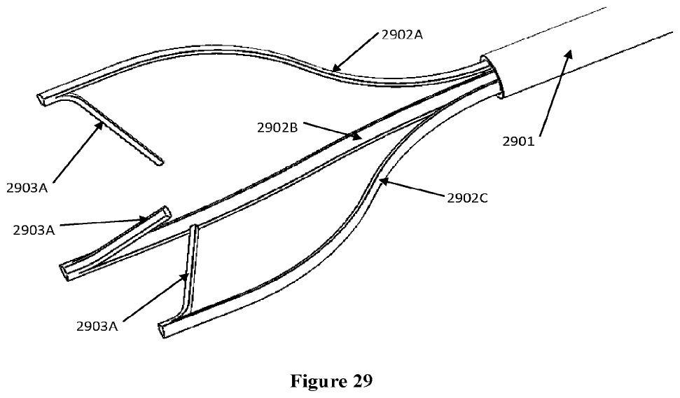

FIG. 29 shows an elongate device 2901 having at least one extensible probe elements 2902A, 2902B, or 2902C. At least one probe element 2902A can be extended or retracted proximally or distally relative to at least one other probe element 2902B. Probe element 2902A is shown with a branch 2903A, which can interact with stationary tissue, movable tissue, or fluid flow in a way that indicates the position of the branch relative to the target tissue. By adjusting the relative positions of two probe elements, 2902A and 2902B, the user can visualize a linear structure in the target tissue, for example a segment of a valve annulus. By adjusting the relative positions of three independent probe elements, 2902A, 2902B, and 2902C, the user can visualize a planar structure in the target tissue, for example a valve annulus.

### EXAMPLES

In one example, an elongate device is attached at the distal end to one or more probe elements. In a further example, the probe elements deflect in contact with bodily tissues. In a further example, the probe elements are radiopaque, making them visible on fluoroscopic examination. In another example, the probe elements include echogenic features. In a further example, the echogenic features are retro-reflective surface textures. In another example, the echogenic features are surface textures that scatter sound waves. In another example, the echogenic features are materials of different densities within the probe elements. In a further example, the echogenic materials are hollow pores contained in the material of the probe element. In another example, the echogenic materials are hollow beads contained in the material of the probe element. In another example, the probe elements are constructed of layers of materials having different densities.

In one example, the probe elements are configured to fold inward to a reduced profile, enabling the elongate device with probe elements to pass through a smaller lumen than when the probe elements are extended. In a further example, the elements fold distally and inward. In another example, the elements fold proximally and inward.

In one example, the elongate device includes an instrument channel for delivering a device to treat, anchor to, mark, or otherwise affect the target tissue. In another example, the instrument channel is centered in the elongate device. In another example, the elongate device contains more than one instrument channel.

In one example, the probe elements are configured to deform as the elongate device tip approaches a section of target tissue. In a further example, this deformation will be visible via one or more imaging modalities (that is, ultrasonography, fluoroscopy, CT scan, MRI, etc.) In a further example, the probe elements flex in response to tissue movement, giving an indication of tissue motion visible on one or more imaging modalities.

In one example, all of the probe elements are substantially the same length. In another example, the elongate device includes probe elements having two or more different lengths. In a further example, one or more probe elements has a long length, and one or more probe elements has a short length. In a further example, probe elements have three or more distinct lengths. In another example, two or more probe elements each have a distinct length. In another example, the elongate device can be rotated relative to the target tissue to bring probe elements of the desired configuration into alignment with the target tissue to refine positioning.

In one example, the probe elements have a substantially consistent cross section along their length. In a further example, the probe elements have one or more sections of reduced cross section. In another example, the probe elements have a cross section that varies along the length of the element.

In one example, one or more probe elements form a single band from the elongated device to the distal end of the element. In another example, one or more probe elements have one or more branches extending off the side creating a second distal or proximal endpoint. In another example, one or more probe elements have one or more branches extending from the end of the probe element. In another example, one or more probe elements have branches extending from the side or end of the probe element and connecting to one or more adjacent probe elements. In another example, two adjacent probe elements are connected at the distal end to allow greater contact with the tissue without substantial increase in mass. In a further example, two adjacent probe elements are connected at the distal end by a foldable branch, which allows the distal ends of the adjacent probe elements to move closer to each other so that they can be delivered through a smaller diameter than in the extended configuration.

In one example, one or more probe elements bend near the junction with the elongated device, and continue in a substantially straight direction to the distal end of the element. In another example, one or more probe elements have a bend disposed at some distance from the junction with the elongated device. In a preferred example, one or more probe elements have a first bend near the junction of the elongated device, and a second bend in substantially the same direction distal to the first bend. In another example, one or more probe elements have a first bend near the junction of the elongated device, and a second bend in substantially the opposite direction distal to the first bend. In another example, one or more probe elements have a continuous bend along a substantial portion of their length.

In one example, one or more probe elements branch to create a probe segment that bends near the branching point. In a preferred example, the probe segment extends inward and distally from the branching point. In another example, the probe segment extends inward and proximally from the branching point. In another example, the probe segment extends outward and distally from the branching point. In another example, the probe segment extends outward and proximally from the branching point.

In one example, each element is coupled to an elongate structure so that the element can be moved or manipulated into position or to a different position. In a further example, the elongate structure comprises suture, wire or the like. In another example each probe element is independently movable.

In one example, one or more probe elements are attached to an elongate structure which is remotely actuated. In another example, two or more elongate structures are independently remotely actuated. In a further example, one or more probe elements include a sensor which can be read remotely.

In one example, an elongate device having at least one hollow channel is attached at the distal end to one or more probe elements. In a further example, the elongate device is a sheath. In a further example, the sheath includes a hemostatic valve. In a further example, the sheath can be steered by controls located outside the body.

In one example, an elongate device having at least one hollow channel is attached at the distal end to one or more probe elements, and an expandable structure is contained within the hollow channel. In a further example, the expandable structure is pushed distally to release it from the elongate device. In a further example, the expandable structure self-expands upon release from the elongate structure. In another example, the expandable structure is a stent. In another example, the expandable structure contains an artificial valve.

In one example, an elongate device is attached to one or more probe elements at the distal end, the elongate device being placed at least partially through an outer elongate device. In a further example, the outer elongate device is a sheath. In a further example, the outer elongate device contains an expandable structure. In a further example, the elongate device with probe elements is disposed at least partially within the expandable structure. In another example, the elongate device with probe elements is disposed alongside the expandable structure.

In one example, an elongate device is attached to one or more probe elements at the distal end, the elongate device being placed at least partially through sheath. In a further example, the sheath contains an implant. In a further example, the elongate device with probe elements is disposed at least partially within the implant. In another example, the elongate device with probe elements is disposed alongside the implant.

In one example, an implant is attached to probe elements. In a further example, the implant has a delivery configuration and an implanted configuration. In a further example, the probe elements deflect to interact with tissue when the implant is in the deployment configuration. In a further example, the probe elements are held against the tissue when the implant is in the implanted configuration. In one example, an elongate device having an instrument channel is attached to one or more probe elements at its distal end, and a tissue coupling anchor is contained at least partially within the instrument channel. In a further example, the elongate device is placed in apposition with target tissue using, while the probe elements aid in visualizing the positional relationship between the target tissue and the elongate device. In a further example, the tissue coupling anchor consists of an implant portion and a delivery portion. In a further example, retracting the tissue coupling anchor proximally brings it proximal to the probe elements. In a further example, retracting the tissue coupling anchor proximally positions the distal end of the tissue coupling anchor within the instrument channel, and extending the tissue coupling anchor distally places the distal tip of the tissue coupling anchor into apposition with the target tissue. In a further example, turning the delivery portion turns the implant portion causing it to helically penetrate the target tissue. In a further example, the delivery portion of the tissue coupling anchor can be detached from the implant portion.

In one example, an elongate device has probe elements attached to its distal end and at least partially contains a tissue shaping template, the elongate device and tissue shaping template are slidably disposed around a tissue coupling anchor which is coupled to the target tissue. In a further example, the elongate device and tissue shaping template are advanced distally over the tissue coupling anchor until the probe elements deflect in contact with the target tissue. In a further example, the elongate device containing the tissue shaping template is rotated about the tissue coupling anchor to align the tissue shaping template with the target tissue. In a further example, the tissue shaping template is coupled to the tissue coupling anchor, and released from the elongate device. In a further example, the elongate device is rotated, advanced and/or retracted so that the probe elements contact the tissue shaped by the tissue shaping template, aiding in visualization of the shaped tissue, and verification of the desired tissue shaping effect.

In one example, an elongate device has an array of probe elements disposed along at least a portion of its length, and the elongate device is placed adjacent to target tissue. In a further example, a first feature of the target tissue deflects a first region of probe elements on the elongate device, indicating the location of this first feature of the target tissue. In a further example, a second feature of the target tissue deflects a second region of probe elements on the elongate device, indicating the location of this second feature of the target tissue as well as the distance between the first feature and the second feature. In a further example, at least one feature of the target tissue is a valve.

In one example, an elongate device has one or more probe elements coupled to its distal end, the elongate device having a cross section which has one lumen, more than one lumen, or no lumens. In a further example, the cross section having no lumens has a shape that is triangular, quadrilateral, pentagonal, hexagonal, heptagonal, octagonal, nonagonal, decagonal, or a polygon having a greater number of sides. In another example, the cross section having no lumens is circular, oval, elliptical, or another predominantly round shape. In another example, the cross section having no lumens has an arcuate shape, and “L” shape, a “C” shape, or comprises a partially open channel.

In one example, an elongate device has one or more probe elements coupled to its distal end and is comprised of two or more elongate sections connected to each other by angled rungs. In a further example, changing the relative position of the elongate sections in a proximal-distal direction changes the height, or width, or diameter of the elongate section.

In one example, an elongate device is coupled to a stationary hub, which is coupled to at least one end of one or more probe elements, another end of at least one of the probe elements being coupled to a movable hub slidably engaged with the elongate device. In a further example, one or more probe elements bend outward, away from the elongate device to form a bulge. In a further example, moving the movable hub towards the stationary hub increases the diameter of the bulge, and moving the movable hub away from the stationary hub decreases the diameter of the bulge. In a further example, the adjustable diameter of the bulge is used to visualize the diameter of a body structure.

In one example, an elongate device is attached to at least one probe element at or near the distal end of the elongate device, the at least one probe element comprising a first material and a second material. In a further example, the difference in properties between the two materials enhances imaging of the probe element. In another example, the different electrical properties between the two materials send information about the conditions in the region of the probe to a display located outside the body. In a further example, the information includes one or more of the following: strain in the probe element, pressure, temperature, electrical conductivity, oxygen saturation. In another example, the second material itself comprises a sensing device capable of sending information to a display located outside the body. In a further example, the information the sensing device sends to the display includes one or more of the following: strain in the probe element, pressure, temperature, electrical conductivity, oxygen saturation. In another example, one of the materials of the probe element is electrically conductive and communicates electrical information such as EKG measurements to a display located outside the body.

In one example, an elongate device is attached to at least one probe element, and an adjustment member is slidably coupled to the elongate device and contacts tone or more probe elements. In a further example, adjusting the proximal to distal position of the adjustment member relative to the probe elements changes the effective length of the probe element. In a further example, moving the adjustment member distally relative to the probe elements makes the effective length of the probe element shorter, and moving the adjustment member proximally relative to the probe elements make the effective length of the probe element longer. In a further example, the effective length of the probe elements is adjusted to a relatively long position during initial positioning of the elongate member relative to the target tissue and adjusted to a relatively shorter position during final positioning, allowing for variable positional precision as needed.

In one example, a first elongate device having one or more probe elements having a first length coupled to its distal end is slidably coupled to a second elongate device having one or more probe elements having a second length coupled to its distal end. In a further example, the probe elements of the first elongate device can be extended distal to the probe elements of the second elongate device or can be retracted proximally to the probe elements of the second elongate device. In a further example, the probe elements of the first elongate device are longer than the probe elements of the second elongate device. In a further example, the first elongate device is arranged to be the distalmost for initial positioning of the coupled elongate devices relative to the target tissue, then the second elongate device is arranged to be the distalmost for precise final positioning of the coupled elongated devices. In a further example, a tissue coupling anchor is slidably coupled to the coupled elongate members and configured to couple to the target tissue once final positioning has been achieved.

In one example, and elongate device contains two or more independently positionable arms having probe elements disposed along their length. In a further example, the arms having probe elements are used to locate a linear structure in the target tissue. In another example, an elongate device contains three or more independently positionable arms having probe elements disposed along their length. In a further example, the three arms having probe elements are used to locate a planar structure in the target tissue. In a further example, the planar structure is a heart valve. In another example, the elongate device is attached to one or more probe elements, and acts as one of the independently controllable arms.

In another example, a device is attached to probe elements. In a still further example, the device is a therapeutic device. In another example, the device is a diagnostic device. In yet another example, the device is a locating or positioning device. In a further example, the device is a sheath with a channel capable of delivering at least one therapeutic, diagnostic, positioning, locating, or marking device.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A surgical locating tool comprising:
a shaft having an engagement end, wherein the shaft is configured to deliver an implant to or engage an interventional tool against an internal tissue surface;
one or more probe elements extending outwardly from the engagement end of the shaft, wherein the probe elements are configured to detectably deflect when engaged against the internal tissue surface.
